(19) United States
(12) Patent Application Publication
(10) Pub. No.: US 2007/0014836 A1
(43) Pub. Date: Jan. 18, 2007

(54) Cologne wipes

(76) Inventor: Kelvin Douglas Hilton, Brooklyn, NY (US)

Correspondence Address: Kelvin D. Hilton, 42 East 57th Street, Brooklyn, NY, 11203-3710, US

(21) Appl. No.: 11/455,528

(22) Filed: Jun. 20, 2006

Related U.S. Application Data
(60) Provisional application No. 60/698,554, filed on Jul. 13, 2005.

(51) Int. Cl. A61K 9/70

(52) U.S. Cl. 424/443

(57) ABSTRACT

Cologne Wipes containers will be constructed of a corrugated cardboard type of material (for FIG. 1 of the drawings, a thin plastic type of material (for FIG. 2 of the drawings), and a thicker type of plastic for FIG. 3 of the drawings). The wipes themselves will be made of a cloth like towelette type of material. The inner linings of the containers will be of a thin plastic sheet type of material. The opening for the mens packaging (FIG. 1 of the drawings) will be that of a velcro type simulated to appear as a zipper. The openings of the portable type (FIG. 2) and the ladies type packaging (FIG. 3 of the drawings) will be an open and close thicker plastic type. The idea of cologne wipes is a very simple and cost effective product to produce and use.

### CROSS-REFERENCE TO RELATED APPLICATION

This nonprovisional utility patent application claims the benefit of filing date Jul. 13, 2005 of provisional application No. 60/698,554.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Cologne Wipes are a personal hygiene product and would most likely be displayed in the personal health care section of the market place. As a personal health care product Cologne Wipes is for the personal use of ones own hygiene. It's the idea of bringing together two well known and highly used products (cologne and wipes). Cologne Wipes will help solve the problems of quickly applying your favorite cologne, having something to freshen up with when needed, portability, convenient, and very useful.

### BRIEF SUMMARY OF THE INVENTION

Cologne Wipes towelettes contains (for the most part) either cologne or perfume and is contained in a moisture sealed package and is used to apply your favorite cologne or perfume directly from the container to the skin. Cologne Wipes are very portable and therefor convenient at times when you need a quick way to freshen up. There are three varieties to choose from as described in the “Detailed Description Of Invention” section below.

### BRIEF DESCRIPTION OF THE SEVERAL VIEWS OF THE DRAWINGS

FIG. 1 shows cologne wipes for men, it comes in a variety of fragrances, the container dimensions are L=7″×W=5″×H=1″ and features a velcro simulated zipper opening on top of the container, container is designed with an inner lining to retain moisture to extend the shelf life of the cologne wipe. Contain 15 wipes.

FIG. 2 shows pocket size portable cologne wipes that features a snap top lid located at top of package. Package dimensions are H=4½″×W=2″×D=1″. Inner lining to retain moisture increasing the life and use of the product. Pocket size cologne wipes available for men and women, and contains 10 wipes. and contains 10 wipes.

FIG. 3 shows cologne (perfume) wipes for women. Inner lining to retain moisture, dimensions are W=5″×4″ and features an open & snap top. Contains 20 wipes.

### DETAILED DESCRIPTION OF THE INVENTION

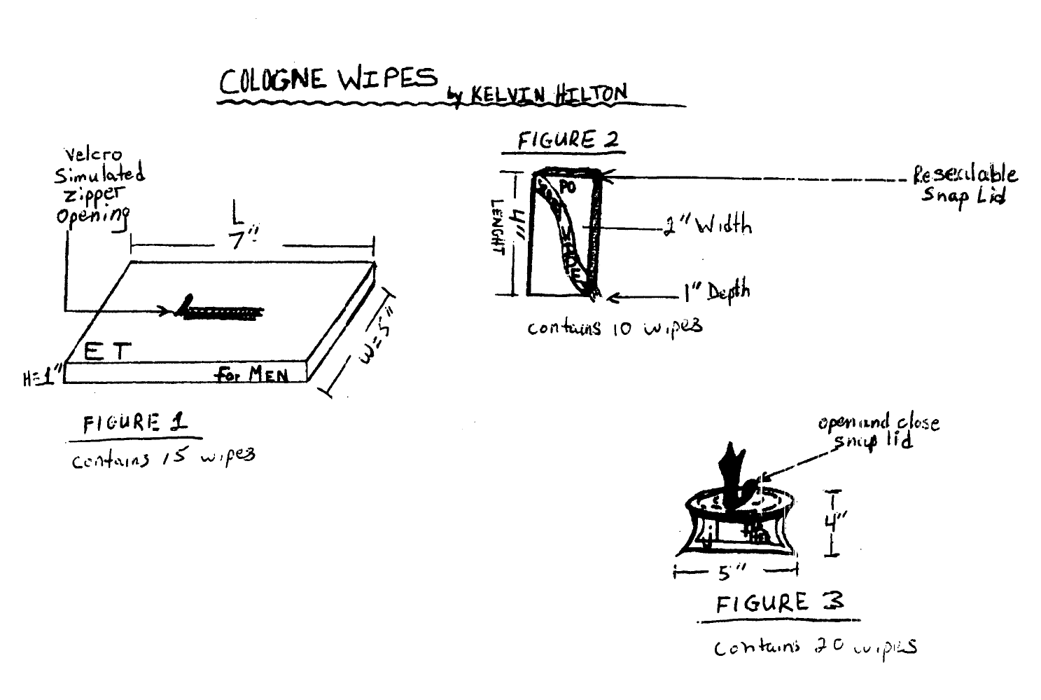

Cologne Wipes are a convenient and portable means of transferring fragrance from containment to skin. Cologne Wipes are a personal care product containing fragrance and alcohol, and may also include other ingredients to comfort and condition the skin (and to also preserve the product). Such ingredients would be purified water, aloe-vera, lanolin, methylisothizolinone and/or methylchorosothiaolinone, fluorocarbon, polyethylene glycol, hydrogenated polyoxyethylene castor oil ethers or polyoxyethlene hardened castor ethers, fatty acids, fatty alcohol. The personal care product will take the form of a durable (2 ply) towelette embodied in a moisture retaining package. There are three distinctive package designs shown in the accompanying drawing. FIG. 1 show the men style (containing any variety of designer mens cologne) that designed to fit in the locker, glove compartment or on the desk. FIG. 2 shows the portable package for either men or women (containing any variety of designer cologne or perfume) and can be carried in ones pocket or pocketbook. FIG. 3 shows the ladies style designed to set on the desk or dresser containing her favorite fragrance There have been many types of wipes on the market, but none actually to market a variety of colognes and perfumes. Also no other wipes product has such a strong towelette (2 ply) at the size of L=9″×W=7½″ allowing the product to be more appliable for better coverage. I would carry out my invention by first selecting the type 2 ply towelettes, select the fragrance (with an agreement from the fragrance designer), select the moisture retaining lining so that there be no need for inner packaging. Add ingredients, assemble product, market and giveaway promos.

1. I claim Cologne Wipes to be my invention, and my invention along.
